(19) United States
(12) Patent Application Publication
(10) Pub. No.: US 2003/0045488 A1
(43) Pub. Date: Mar. 6, 2003

(54) Antisense oligonucleotides comprising universal and/or degenerate bases

(76) Inventors: Bob D. Brown, Encinitas, CA (US); Timothy A. Riley, San Diego, CA (US)

Correspondence Address: KNOBBE MARTENS OLSON & BEAR LLP, 2040 MAIN STREET, FOURTEENTH FLOOR, IRVINE, CA, 91614, US

(21) Appl. No.: 09/931,732

(22) Filed: Aug. 16, 2001

Related U.S. Application Data
(63) Continuation of application No. PCT/US00/09293, filed on Apr. 7, 2000.
(60) Provisional application No. 60/128,377, filed on Apr. 8, 1999.

(51) Int. Cl. A61K 48/00; C07H 21/04

(52) U.S. Cl. 514/44; 536/23.2

(57) ABSTRACT

Antisense oligonucleotides containing one or more degenerate and/or universal bases, and one or more modified backbone linkages, and use of these oligonucleotides for cleaving target RNA molecules.

### CROSS-REFERENCE TO RELATED APPLICATIONS

This application is a continuation of International Application number PCT/US00/09293 and claims the benefit of priority of International Application No. PCT/US00/09293 having international filing date Apr. 7, 2000, designating the United States of America and published in English, which claims the benefit of priority of U.S. Application Ser. No. 60/128,377 filed Apr. 8, 1999; both of which are hereby expressly incorporated by reference in their entireties.

### FIELD OF THE INVENTION

The present invention relates to antisense oligonucleotide compositions comprising one or more universal and/or degenerate bases, and to methods for using these oligonucleotides to target RNA molecules

### DESCRIPTION OF THE RELATED ART

Antisense technology is based on the finding that gene expression can be modulated using an oligonucleotide which binds to the target RNA. By exploiting the Watson-Crick base pairing and the ability to recruit certain nucleases, particularly RNase H, to specifically cleave the target RNA in the DNA/RNA hybrid, one can design antisense molecules which are highly specific for the target nucleic acid molecule. However, there are families of genes in which this high degree of specificity may be detrimental. For example, it may be desirable to target two or more of these genes if there is a synergistic effect if the genes are inactivated together.

Typical antisense compounds are modified nucleic acids that bind to their target RNA via Watson-Crick base pairing. Different constructions can recruit a variety of RNases to mediate the cleavage of the target RNA. The most common RNase is RNase H which recognizes a DNA/RNA duplex, followed by cleavage of the target RNA. The oligonucleotide most commonly used for this purpose contains unmodified (naturally-occurring) bases (A, T, G, C) and a modified backbone called a phosphorothioate which renders the oligonucleotide resistant to nucleases. Other backbone modifications such as 2′-O-alkyl render the oligonucleotide unable to mediate RNase H cleavage of the target RNA. There are many reports of the combination of non-RNase H substrate portions and RNase H substrate portions within a single antisense oligonucleotide. These non-RNase H substrate portions provide both binding and specificity for the antisense oligonucleotide. Examples of these backbones include methylphosphonates, morpholinos, MMI, peptide nucleic acids (PNA) and 3′-amidates. Sugar modifications that increase antisense oligonucleotide binding and nuclease stability include 2′-O-alkyl, 2′-O-allyl, 2′-O-methoxyethyl, 2′-O-alkylaminoalkyl, 2′-fluoro (2′-F) and 2′-amino.

Universal or degenerate bases are heterocyclic moieties which have the ability to hydrogen bond to more than one base in a DNA duplex without destroying the ability of the whole molecule to bind to the target. The use of oligonucleotides having unmodified backbones and containing degenerate or universal bases is known in the PCR primer literature (Bergstrom et al., J. Am. Chem. Soc. 117:1201-1209, 1995; Nichols et al., Nature 369:492-493, 1994; Loakes, Nucl. Acids Res. 22:4039-4043, 1994; Brown, Nucl. Acids Res. 20:5149-5152, 1992). However, to date these universal and degenerate bases have not been used in antisense technology, and have not been incorporated into oligonucleotides which comprises modified backbone linkages. The present invention addresses these antisense compositions and methods.

### SUMMARY OF THE INVENTION

One embodiment of the present invention is an antisense oligonucleotide having at least one non-naturally occurring backbone linkage and having between 6 and about 50 bases, wherein at least one of the bases are universal and/or degenerate bases. Preferably, no more than about 50% of the bases are universal and/or degenerate bases.

Another embodiment of the present invention is an antisense oligonucleotide comprising a first non-RNase H recruiting region having between 3 and about 15 bases, an RNase H recruiting region having between 3 and about 15 bases, and a second non-RNase H recruiting region, wherein at least one of the bases are universal and/or degenerate bases. Preferably, no more than about 50% of the bases are universal and/or degenerate bases.

The present invention also provides an antisense oligonucleotide comprising a non-RNase H recruiting section and an RNase H recruiting section, wherein at least one but of the bases are universal and/or degenerate bases. Preferably, no more than about 50% of the bases are universal and/or degenerate bases.

Another embodiment of the present invention is an oligonucleotide comprising an RNase L-recruiting region comprising a 2′-5′ adenosine oligomer, wherein at least one of the bases in the RNA targeting region of the oligonucleotide are universal and/or degenerate bases. Preferably, not more than about 50% of the bases in the RNA targeting region are universal and/or degenerate bases.

The present invention also provides an oligonucleotide designed to recruit RNase P, wherein at least one of the bases in the RNA targeting region of the oligonucleotide are universal and/or degenerate bases. Preferably, no more than about 50% of the bases in the RNA targeting region are universal and/or degenerate bases.

Another embodiment of the present invention is a ribozyme having at least one universal and/or degenerate base in its RNA targeting region. Preferably, no more than about 50% of the bases in the RNA targeting region are degenerate and/or universal bases.

The present invention also provides a method for cleaving a target RNA molecule, comprising the step of contacting the RNA molecule with any of the oligonucleotides described above in the presence of an RNase activity capable of cleaving the target. Preferably, the RNase is RNase H, RNase L or RNase P.

The present invention also provides a method for cleaving a target RNA molecule, comprising the step of contacting the RNA molecule with the ribozyme described above.

The present invention also provides a method for cleaving a target RNA molecule, comprising the step of contacting said RNA molecule with the ribozyme described above.

Another embodiment of the present invention is a method for cleaving a target RNA molecule, comprising the step of contacting the RNA molecule with an oligonucleotide having between 6 and about 50 bases, wherein the oligonucleotide comprises at least one universal and/or degenerate base.

The present invention also provides a method for reducing the deleterious effects of an antisense oligonucleotide comprising one or more sequence motifs, comprising replacing one or more bases within said one or more sequence motifs with one or more universal and/or degenerate bases. Preferably, the sequence motif is a CG dinucleotide. In another aspect of this preferred embodiment, the sequence motif is a poly-G sequence.

### BRIEF DESCRIPTION OF THE DRAWINGS

FIG. 1 shows a sequence alignment of a region of high homology between the human bcl-2A and human bcl-xL genes. Antisense oligonucleotides complementary to the aligned sequence region, and which include one or more universal and/or degenerate bases, are shown below the sequence alignment. Base mismatches are indicated by asterisks. B indicates a universal base. P and K are degenerate bases which pair with any pyrimidine and any purine, respectively.

FIG. 2 shows a sequence alignment of three homology regions of three human protein kinase C (PKC) family members. Antisense oligonucleotides complementary to the aligned sequence region, and which include one or more universal and/or degenerate bases, are shown below the sequence alignment. These antisense oligonucleotides simultaneously target two or more PKC family members.

FIG. 3 shows a sequence alignment of homology regions between two alleles of the bcl-2 gene, bcl-2B and bcl-2C. Representative antisense oligonucleotides including one or more universal and/or degenerate bases are shown below the sequence alignments.

### DETAILED DESCRIPTION OF THE PREFERRED EMBODIMENTS

The present invention provides antisense oligonucleotides including one or more universal and/or degenerate bases and methods for targeting RNA which includes a region complementary or nearly complementary to the antisense oligonucleotides. Conventional antisense oligonucleotide containing only naturally occurring nucleotide bases (A, T, G, C, and U) are efficient only when they are completely complementary to their target sequence. In other words, the oligonucleotide cannot bind with sufficient affinity to mismatched oligonucleotides. This compromises the ability of conventional oligonucleotides to bind to single nucleotide polymorphisms (SNPs), and does not permit targeting of two or more homologous genes containing one or more mismatches with a single antisense oligonucleotide. The present invention solves this problem by incorporating one or more universal and/or degenerate bases (defined below) into antisense oligonucleotides. Because these universal and/or degenerate bases can tolerate nucleotide mismatches and bind with sufficient affinity to allow recruitment of nucleases, they solve this mismatch problem.

The incorporation of at least one universal and/or degenerate base into an antisense oligonucleotide can be used to reduce or eliminate the deleterious effects caused by a series or group of natural bases. Various short base sequences in oligonucleotides cause significant sequence-dependent biological effects which are not antisense-specific. For example, almost all nucleotides containing an unmethylated “CG” dinucleotide cause a variety of immune-activation effects when injected into animals, or when incubated with isolated bone marrow cells. The most common immune activation effects are enhanced B-cell proliferation and cytokine production, including inflammatory cytokines such as interleukin-2. This immune activation phenomenon is believed to be responsible for some deleterious side effects of many therapeutic antisense oligonucleotide candidates. The present invention addresses this problem by the substitution of a degenerate or universal base for C or G in these “CG” repeats. This is believed to eliminate undesirable immune activation effects, while maintaining efficient, specific antisense activity.

In addition, “GGGG” and other poly-G motifs have been shown repeatedly to produce non-antisense effects such as growth inhibition in cell cultures and high systemic toxicity in animals. Substitution of universal and/or degenerated bases within tetra-G or other poly-G motifs can “break-up” these sequences and result in an antisense oligonucleotide having significant research and therapeutic utility in both animals and cell culture.

The term “antisense” as used herein refers to a molecule designed to interfere with gene expression and capable of recognizing or binding to a specific desired target polynucleotide sequence. Antisense molecules typically (but not necessarily) comprise an oligonucleotide or oligonucleotide analog capable of binding specifically to a target sequence present on an RNA molecule. Such binding interferes with translation by a variety of means, including preventing the action of polymerases, RNA processing and recruiting and/or activating nucleases such as RNase H, RNase L and RNase P.

The term “ribozyme” as used herein refers to an oligonucleotide or oligonucleotide analog capable of catalytically cleaving a polynucleotide.

The term “oligonucleotide” refers to a molecule consisting of DNA, RNA or DNA/RNA hybrids.

The term “oligonucleotide analog” refers to a molecule comprising an oligonucleotide-like structure, for example having a backbone and a series of bases, wherein the backbone and/or one or more of the bases can be other than the structures found in naturally-occurring DNA and RNA. “Non-natural” oligonucleotide analogs include at least one base or backbone structure that is not found in natural DNA or RNA. Exemplary oligonucleotide analogs include, but are not limited to, DNA, RNA, phosphorothioate oligonucleotides, peptide nucleic acids (PNA), methoxyethyl phosphorothioates, oligonucleotide containing deoxyinosine or deoxy 5-nitroindole, and the like.

The term “backbone” as used herein refers to a generally linear molecule capable of supporting a plurality of bases attached at defined intervals. Preferably, the backbone will support the bases in a geometry conducive to hybridization between the supported bases of a target polynucleotide.

The term “non-naturally occurring base” refers to a base other that A, C, G, T and U, and includes degenerate and universal bases as well as moieties capable of binding specifically to a natural base or to a non-naturally occurring base. Non-naturally occurring bases include, but are not limited to, propynylcytosine, propynyluridine, diaminopurine, 5-methylcytosine, 7-deazaadenosine and 7-deazaguanine.

The term “universal base” refers to a moiety that may be substituted for any base. The universal base need not contribute to hybridization, but should not significantly detract from hybridization. Exemplary universal bases include, but are not limited to, inosine, 5-nitroindole and 4-nitrobenzimidazole.

The term “degenerate base” refers to a moiety that is capable of base-pairing with either any purine, or any pyrimidine, but not both purines and pyrimidines. Exemplary degenerate bases include, but are not limited to, 6H, 8H-3,4-dihydropyrimido[4,5-c][1,2]oxazin-7-one (“P”, a pyrimidine mimic) and 2-amino-6-methoxyaminopurine “K”, a purine mimic).

The term “target polynucleotide” refers to DNA, for example as found in a living cell, with which the antisense molecule is intended to bind or react.

The term “activity” refers to the ability of an antisense molecule of the invention, when hybridized to a target polynucleotide, to interfere with the transcription and/or translation of the target polynucleotide. Preferably, the interference arises because the antisense molecule, when hybridized, recruits a nuclease, and/or serves as a nuclease substrate. The term “interference” includes inhibition to any detectable degree.

The term “RNase H recruiting” refers to an oligonucleotide having at least one phosphorothioate and/or phosphodiester backbone. This type of backbone is recognized by RNase H once a RNA/DNA hybrid is formed and allows RNAse H to cleave the target RNA.

The term “non-RNase H-recruiting” refers to an oligonucleotide having linkages other than deoxyphosphodiester or deoxyphosphorothioate linkages, including, but not limited to, 2′-O-alkyl, PNA, methylphosphonate, 3′-amidate, 2′-F, morpholino, 2′-O-alkylaminoalkyl and 2′-alkoxyalkyl. This type of oligonucleotide is not recognized by RNase H after formation of a DNA/RNA hybrid.

The term “RNase L recruiting” refers to an oligonucleotide comprising four consecutive adenosine bases in 2′, 5′-linkage which form an oligomer. This oligomer is recognized by RNase L once a DNA/RNA hybrid is formed (See U.S. Pat. No. 5,583,032).

The term “RNase P recruiting” refers to an oligonucleotide capable of forming a stem-loop structure which is recognized by RNase P, an enzyme normally involved in generation of mature tRNA by cleaving a portion of tRNA precursor molecules. This stem-loop structure resembles the native tRNA substrate and is described by Ma et al. (Antisense Nucl. Acid Drug Dev. 8:415-426, 1998) and in U.S. Pat. No. 5,877,162.

The antisense oligonucleotides and oligonucleotide analogs of the invention are preferably between 6 and about 50 bases long, more preferably between about 10 and 30 bases long, and most preferably between about 15 and 25 bases long. Oligonucleotides having 18 base pairs are particularly preferred.

The antisense oligonucleotides and oligonucleotide analogs of the invention typically contain at least one universal or degenerate base, and at least one modified backbone linkage. In general, these oligonucleotides do not contain more than about 50% universal and/or degenerate bases.

The oligonucleotides and oligonucleotide analogs of the present invention can be synthesized using standard oligonucleotide synthesis methods (see Example 1).

The oligonucleotides used in the binding domains can employ any any backbone and any sequence capable of resulting in a molecule that hybridizes to natural DNA and/or RNA. Examples of suitable backbones include, but are not limited to, phosphodiesters and deoxyphosphodiesters, phosphorothioates and deoxyphosphorothioates, 2′-O-substituted phosphodiesters and deoxy analogs, 2′-O-substituted phosphorothioates and deoxy analogs, morpholino, PNA (U.S. Pat. No. 5,539,082), 2′-O-alkyl methylphosphonates, 3′-amidates, MMI, alkyl ethers (U.S. Pat. No. 5,223,618) and others as described in U.S. Pat. Nos. 5,378,825, 5,489,677, 5,541,307, and the like. Where RNase activity is desired, a backbone capable of serving as an RNase substrate is employed for at least a portion of the oligonucleotide.

Universal bases suitable for use in the present invention include, but are not limited to, deoxy 5-nitroindole, deoxy 3-nitropyrrole, deoxy 4-nitrobenzimidazole, deoxy nebularine, deoxyinosine, 2′-OMe inosine, 2′-OMe 5-nitroindole, 2′-OMe 3-nitropyrrole, 2′-F inosine, 2′-F nebularine, 2′-F 5-nitroindole, 2′-F 4-nitrobenzimidazole, 2′-F 3-nitropyrrole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, 2′-O-methoxyethyl 3-nitropyrrole, deoxy Rₚ MP-5-nitroindole dimer 2′-OMe Rₚ MP-5-nitroindole dimer and the like.

Degenerate bases suitable for use in the present invention include, but are not limited to, deoxy P (A&G), deoxy K (U&C), 2′-OMe 2-aminopurine (U&C), 2′-OMe P (G&A), 2′-OMe K (U&C), 2′-F-2-aminopurine (U&C), 2′-F P (G&A), 2′-F K (U&C), PNA-2-aminopurine (U&C), PNA-P (G&A), PNA-K (U&C), morpholino-2-aminopurine (U&C), morpholino-P (G&A), morpholino-K (U&C), phosphoramidate-2-aminopurine (C&U), phosphoramidate-P (G&A), phosphoramidate-K (U&C), 2′-O-methoxyethyl 2-aminopurine (U&C), 2′-O-methoxyethyl P (G&A), 2′-O-methoxyethyl K (U&C), deoxy Rₚ MP-KP dimer, deoxy Rₚ MP-PK dimer, deoxy Rₚ MP-Kk dimer, deoxy Rₚ MP-PP dimer, 2′-OMe Rₚ MP-KP dimer, 2′-OMe Rₚ MP-PK dimer, 2′-OMe Rₚ MP-KK dimer, 2′-OMe Rₚ MP-PP dimer and the like.

The present invention provides methods for use of universal and/or degenerate bases in antisense oligonucleotides to provide single antisense molecules that target more than one gene. These universal and/or degenerated bases can be used in either the RNase H portion or non-RNase H portion of antisense molecules. The ability to bind to more than one base on a target provides the flexibility of making one antisense molecule that targets more than one RNA sequence.

Oligonucleotide synthesis is well known in the art, as is synthesis of oligonucleotides containing modified bases and backbone linkages. In one embodiment of the present invention, there is provided an antisense phosphorothioate oligonucleotide having between 6 and about 50 bases in which at least one of its bases are replaced with universal and/or degenerate bases. In a preferred embodiment, no more than about 50% of the bases are universal and/or degenerate bases. Another oligonucleotide for use in the present invention comprises a non-RNase recruiting portion of between 3 and about 15 bases, followed by an RNase-recruiting portion of between 3 and about 15 bases, followed by a second non-RNase H-recruiting portion of 3 to about 15 bases, wherein at least one of the bases contained in the oligonucleotide are degenerate and/or universal bases. In a preferred embodiment, no more than about 50% of the bases are universal and/or degenerate bases. Another antisense oligonucleotide contemplated for use in the present invention comprises a non-RNase H recruiting portion followed by a RNase H-recruiting portion in which at least one of its bases are replaced with universal and/or degenerate bases. In a preferred embodiment, no more than about 50% of the bases are universal and/or degenerate bases. An antisense oligonucleotide comprising an RNase H-recruiting portion followed by a non-RNase H-recruiting portion, in which at least one of its bases are replaced with degenerate and/or universal bases, is also within the scope of the present invention. In a preferred embodiment, no more than about 50% of the bases are universal and/or degenerate bases.

Other antisense oligonucleotides contemplated for use in the present invention include: an oligonucleotide comprising an RNase L recruiting oligonucleotide 2′-5′ adenosine moiety in which the oligonucleotide comprises at least one degenerate and/or universal base; and an oligonucleotide designed to recruit RNase P in which the oligonucleotide comprises at least one degenerate and/or universal base. In a preferred embodiment, no more than about 50% of the bases are universal and/or degenerate bases.

Another embodiment of the invention is a ribozyme in which at least one base within the RNA targeting sequence is a degenerate and/or universal base. In a preferred embodiment, no more than about 50% of the bases are universal and/or degenerate bases. The minimum sequence requirements for ribozyme activity are described by Benseler et al. (J. Am. Chem. Soc. 115:8483-8484, 1993). Hammerhead ribozyme molecules comprise end domains “I” and “III”) which hybridize to the substrate polynucleotide, a catalytic portion, and a stem loop structure “II”) which can be substituted by a variety of other structures capable of holding the molecule together.

The antisense oligonucleotides of the present invention can be used to target one or more genes, more preferably therapeutic genes, and most preferably anti-apoptosis or chemoresistance genes as described in the examples presented below.

Representative classes of antisense oligonucleotides for use in the present invention are shown below. Although this figure shows 18-mers, this should be considered illustrative rather than limiting.

| | |
|---|---|
| 5′-NNN NNN BBB BBB NNN NNN-3′ | (SEQ ID |
| | NO:1) |
| 5′-NNN NNN BBB BBB NNN NNN-3′ | (SEQ ID |
| | NO:2) |
| 5′-NNN NNN BBB BBB NNN NNN-3′ | (SEQ ID |
| | NO:3) |
| 5′-NNN NNN BBB BBB NNN NNN-3′ | (SEQ ID |
| | NO:4) |
| 5′-NNN BNN BBN BNB NBN NBN-3′ | (SEQ ID |
| | NO:5) |
| 5′-NNN BNN BBN BNB NBN NBN-3′ | (SEQ ID |
| | NO:6) |
| 5′-NNN BNN BBN BNB NBN NBN-3′ | (SEQ ID |
| | NO:7) |
| 5′-a*a*a*a*-----NNN BNN BBN BNB NBN NBN-3′ | (SEQ ID |
| | NO:8) |
| 5′-NNN BNN BBN#BNB NBN NBN-3′ | (SEQ ID |
| | NO:9) |
| 5′-NNN BNN BBN&BNB NBN NBN-3′ | (SEQ ID |
| | NO:10) |
| 5′-NNN BNN BBN BNB NBN NBN-3′ | (SEQ ID |
| | NO:11) |

In these sequences, B is a universal base or degenerate base; N is a natural or non-naturally occurring base capable of specific recognition of an RNA target base including, but not limited to, A, C, G, T, U, propynyl C, propynyl U, diamopurine, 5-MeC, 7-deaza A and 7-deaza G. The underline represents the non-RNase H recruiting section, including, but not limited to, 2′-O-alkyl, PNA, methylphosphonate, 3′-amidate, 2′-F, morpholino, 2′-O-alkylaminoalkyl and 2′-alkoxyalkyl. The “- - - -” represents a linker including, but not limited to the one disclosed in U.S. Pat. No. 5,583,032. The “#” represents the ribozyme cleaving portion of a ribozyme oligonucleotide; the “&” represents the stem loop structure that recruits RNase P; and a*a*a*a* represents a tetramer of oligomeric 2′-5′ adenosine. SEQ ID NO: 11 is also designed to recruit RNase P by inducing formation of a loop structure on the target RNA which is a substrate for RNase P (See U.S. Pat. No. 5,877,162).

The antisense oligonucleotides and ribozymes described above are used to cleave one or more target RNA molecules in vitro or in vivo.

### Example 1

### Oligonucleotide Synthesis

All reagents are used dry (<30 ppm water). Oligonucleotide synthesis reagents are purchased from Glen Research. Amidites in solution are dried over Trap-paks (Perkin-Elmer Applied Biosystems, Norwalk, Conn.). A solid support previously derivatized with a dimethoxy trityl (DMT) group protected propyl linker is placed in a DNA synthesizer column compatible with a Perkin-Elmer Applied Biosystems Expedite synthesizer (1 mmol of starting propyl linker). The DMT group is removed with a deblock reagent (2.5% dichloroacetic acid in dichloromethane). The standard protocols for RNA and DNA synthesis are applied to amidites (0.1 M in dry acetonitrile). The amidites are activated with tetrazole (0.45 M in dry acetonitrile). Coupling times are typically up to 15 minutes depending on the amidite. The phosphonite intermediate is treated with an oxidizing Beaucage sulfurizing reagent. After each oxidation step, a capping step is performed which places an acetyl group on any remaining uncoupled 5′-OH groups by treatment with a mixture of two capping reagents: CAP A (acetic anhydride) and CAP B (n-methylimidazole in THF). The cycle is repeated a sufficient number of times with various amidites to obtain the desired sequence. After the desired sequence is obtained, the support is treated at 55° C. in concentrated ammonium hydroxide for 16 hours. The solution is concentrated on a speed vac and the residue is taken up in 100 ml aqueous 0.1 M triethylammonium acetate. This is applied to an HPLC column (C-18, Kromasil, 5 mm, 4.3 mm diameter, 250 mm length) and eluted with an acetonitrile gradient (solvent A, 0.1 M TEM; solvent B, 0.1 M TEAA and 50% acetonitrile) over 30 minutes at 1 ml/min flow rate. Fractions containing greater than 80% pure product are pooled and concentrated. The resulting residue is taken up in 80% acetic acid in water to remove the trityl group and reapplied to a reverse phase column and purified as described above. Fractions containing greater than 90% purity are pooled and concentrated.

The antisense activity of the oligonucleotides of the invention can be determined by standard assay methods as described, for example, in Examples 2-4. In general, one can prepare a target polynucleotide having a known sequence, contact the target with oligomers of the invention selected to bind the target sequence to form a complex, subject the complex to cleavage with the desired target nuclease and analyze the products to determine if cleavage occurred. The activity can be determined by detecting cleaved target polynucleotides directly (e.g., by hybridization to a labeled probe, amplification by PCR, visualization on a gel, and the like), or by an effect on a host cell phenotype (for example, expression or lack of expression of a selected protein). The RNase H cleavage assay is described below

### Example 2

### RNase H Cleavage Assay

PCR is used to prepare a dsDNA fragment encoding part of secreted alkaline phosphatase (SEAP) using the following primers:

| | |
|---|---|
| P3-5′-CGAAA-TTAAATCGACTCACTAT-3′, | (SEQ ID NO:12) |
| P3.1-3′-GCTTTAATTATGCTGAGTGATATCCCGAAGCTTAGCGCTTAAGCGGGTGGT- | (SEQ ID NO:13) |
| ACGACGACGACGACGACGACGACCCGGAC-5′; | |
| P4-3′-TAGGGTCAACTCCTCCTCTTGG-5′; and | (SEQ ID NO:14) |
| P5-3′-TACGAC-GACGACGACGACGACGACCCGGACTCCGATGTCGAGAGGGACCCGTAGTA- | (SEQ ID NO:15) |
| GGGTCAACTCCTCGTCTTGG-5′. | |

These primers are based on the SEAP RNA fragment (1 to 102) having the sequence: 5′-GGGCTTCGMTCGCGAATTCGCCCACCATGCTGCTGCTGCTGCTGCTGGGCCTGAGGCTACA GCTCTCCCTGGGCATCATCCCAGTTGAGGAGGAGAACC-3′ (SEQ ID NO: 16).

PCR amplification is performed under the manufacturer's (Life Technologies) recommendation reaction conditions. Primers P3.1 and P5 are used at 10 nM, while primers P3 and P4 are used at 0.50 μM. The PCR program is 94° C. for 5 minutes, 35 cycles at 52° C. for 30 seconds, 72° C. for 1 minute, 94° C. for 45 seconds and 72° C. for 10 minutes.

SEAP dsDNA is then transcribed into ssRNA using a RiboMax) large scale RNA kit (Promega, Madison, Wis.). The SEAP DNA concentration is 30 μg/ml. The transcription reaction is terminated by adding DNase I and incubating at 37° C. for 15 minutes. DNA fragments and free nucleotides are removed by precipitation in ethanol/sodium acetate and washing with 70% ethanol. The RNA was suspended and diluted to approximately 2 μM for use in the RNase H activity assays.

Oligonucleotides of the present invention complementary to a portion of SEAP RNA (20 μM each), SEAP RNA (10 μl of 2 μM solution), and Tris/EDTA buffer (10 mM Tris-HCl, pH 7.4, 1 mM EDTA, “TE”, qs to 2 μl) are added to 500 μl thin-wall reaction tubes and incubated for 3 to 5 minutes at 40° C. to reach thermal equilibrium. RNase H buffer (10×: 200 mM Tris-HCl, pH 7.4-7.5, 1,000 mM KCl, 100 mM MgCl₂.6H₂O, 0.5 mM dithiothreitol, 25% w/v sucrose), RNase H (0.4 to 0.6 U, Promega), and water (qs to 20 μl), are combined to form a cocktail, and incubated for 3 to 5 minutes at 40° C. Then, 8μl of the cocktail is added to each reaction tube and mixed as quickly as possible to prevent cooling. Reactions are incubated at 40° C. for 30 minutes in an MJ Research (Watertown, Mass.) PCT-100 temperature controller. Reactions are stopped by adding 20μl FDE sample buffer (90% v/v formamide, 10% v/v 10×TBE buffer, 0.5% w/v bromphenol blue, 25 mM EDTA) (1×TBE: 89 mM Tris base, 89 mM boric acid, 2 mM EDTA, pH 8.0) to each reaction and heating to 90° C. for 3 to 5 minutes.

Each sample (8 to 10 μl) is subjected to polyacrylamide gel electrophoresis on denaturing 15% gels at 200 volts for about one hour, or until the dye front reaches the bottom of the gel. Nucleic acid bands in gels are visualized by soaking the gels in a 1:10,000 dilution of Cyber Gold™ (Molecular Probes, Junction City, Oreg.) in 1×TBE for 5-10 minutes, soaking in 1×TBE for an additional 5-10 minutes and irradiating on a short wave UV transilluminator. The results are recorded by photographing the CyberGold™ fluorescence using a CyberGREEN™ filter and a Polaroid MP-4 camera with Polaroid Type 667 3000 ASA black and white film.

Duplex DNA ladders (20 bp and 100 bp, GenSura, San Diego) are used as size standards. Standard ladders are not heated before loading onto gels, and are undenatured, running as duplex DNA fragments in both denaturing and non-denaturing gels.

### Example 3

### Intracellular Antisense Activity Against Protein Kinase C alpha (PKCα)

Protein kinase C alpha (PKCα) is used as a gene target to demonstrate antisense activity of the oligonucleotides comprising degenerate and/or universal bases of the invention. PKCα is a normal human gene that is overexpressed in a majority of human cancer types, and is one of the most highly publicized of all antisense target genes.

A human bladder carcinoma cell line (T-24, ATCC HTB-4) , a cell line known to overexpress PKCα, is cultured using standard methods: 37° C., 5% CO₂ in 75 cm² flasks in McCoy's 5A medium (Mediatech, Herndon, Va.) with 10% fetal bovine serum and penicillin-streptomycin. For antisense experiments, T-24 cells are plated into 12-well plates. at 75,000 cells/well and allowed to adhere and recover overnight before transfection. The oligonucleotide 5′-GTTCTCXXXXXXGAGTTT-3′ (SEQ ID NO: 17) in which the X residues are universal and/or degenerate bases (the same or different), and in which remaining residues are connected by modified backbone linkages other than phosphorothioate linkages, and a control oligonucleotide, are transfected into T-24 cells using a cationic lipid-containing cytofection agent (LipofectACE™) (GibcoBRL, Gaithersburg, Md.) which provides efficient nuclear delivery of fluorescently labeled oligonucleotides of the invention in T-24. This is an analog of 5′-GTTCTCGCTGGTGAGTTTCA-3′ (SEQ ID NO: 18) which is a known PKCα antisense molecule.

Oligonucleotides of the invention and conventional all-phosphorothioate oligonucleotides are diluted into 1.5 ml of reduced serum medium Opti-MEM® I (GibcoBRL) to a concentration of 400 nM each. The oligonucleotide-containing solutions are then mixed with an equal volume of OPti-MEM I containing LipofectACE sufficient to give a final lipid to oligonucleotide ratio of 5 to 1 by weight. The final concentration of oligonucleotide is 200 nM. The oligonucleotide/lipid complexes are incubated at room temperature for 20 minutes before adding to tissue culture cells.

Cells are washed once in phosphate buffered saline (PBS) to rinse away serum-containing medium, followed by addition of 1 ml transfection mix to each well of a 12-well plate. All transfections are performed in triplicate. The cells are allowed to take up oligonucleotide/lipid complexes for 22 hours prior to harvesting the total cellular RNA. Mock transfections consist of cells treated with Opti-MEM 1 only.

After 22 hours of antisense treatment, total RNA is harvested from the cells. The cells are released from the plates by trypsin/EDTA treatment according to standard methods. The triplicate groups of cells are pooled and total cytoplasmic RNA is isolated using an RNeasy kit (QIAGEN) according to the manufacturer's protocols. The RNA is treated with DNase I and UV quantitated according to standard methods.

Reverse transcriptase/polymerase chain reaction (RT-PCR) is performed with the methods and materials from a SuperScript One-Step RT-PCR kit from GibcoBRL. The RT-PCR reactions to detect PKCα are performed in two independent runs, with PKCα-specific primers from Oxford Biomedical Research and 100 ng of input total RNA.

Control multiplex RT-PCRs (MP RT-PCRs) are performed to confirm equal quantities of input RNA into each PKCα RT-PCR. The primers, reagents and protocol are from Maxim Biotech. The control MP RT-PCRs amplify BAX and LICE genes equally in all samples, confirming that equal amounts of intact RNA are added to the PKCα RT-PCRs.

All RT-PCR reactions are performed according to the following program of a PTC-1000 thermocycler (MJ Research): Step 1, 50° C. for 35 minutes; Step 2, 94° C. for 2 minutes; Step 3, 55° C. for 30 seconds; Step 4; 72° C. for 1 minute; Step 5, 94° C. for 30 seconds; Step 6, go to step 3, 33 more times; Step 7, 72° C. for 10 minutes; Step 8, end. all RT-PCR products are separated on a 4% Super Resolution Agarose TBE gel (Apex) and stained with Cyber Gold™ according to the manufacturer's instructions. Gels are photographed on Polaroid Type 667 film.

### Example 4

### Antisense Activity Against Human Bcl2 Gene in Tissue Culture Cells

B cell lymphoma-associated gene 2 (Bcl2) is a “normal” human gene that is overexpressed in a majority of human cancer types. The Bcl2 protein regulates cell death and BCI overexpression is known to cause cells to be chemotherapy and radiation resistant. The following Bcl2-targeted antisense molecule is synthesized: 5′-TCTXCCXXCXTXCXCCXT-3′ (SEQ ID NO: 19), in which X is the same or different universal and/or degenerate bases, and in which the first nine residues are a non-RNase H recruiting region (i. e., contain modified backbone linkages other than phosphorothioate linkages). This is an analog of the oligonucleotide 5′-TCTCCCAGCGTGCGCCAT-3′ (SEQ ID NO: 20).

T-24 cells are plated at 75,000 cells/well and allowed to adhere and recover overnight before oligonucleotide transfections. Test and control oligonucleotides are transfected into T-24 cells using LipofectACE™. Oligonucleotides are diluted into 1.5 ml of reduced serum medium (OptiMEM™, GibcoBRL) to a concentration of 400 nM each. The oligonucleotide-containing solutions are then mixed with an equal volume of Opti-MEM I containing LipofectACE sufficient to five a final lipid to oligonucleotide ratio of 5 to 1 by weight. the final concentration of oligonucleotide is 200 nM. The oligonucleotide/lipid complexes are incubated at room temperature for 20 minutes before adding to tissue culture cells. Cells are washed once in PBS , followed by addition of 1 ml of transfection mixed into each well of a 12-well plate. All transfections are performed in triplicate. Cells are allowed to take up oligonucleotide/lipid complexes for 24 hours prior to harvesting of total cellular RNA. Mock transfections consist of cells treated with OPti-MEM I only. Total cytoplasmic RNA is isolated and quantitated as described in Example 3.

RT-PCR is performed as described in Example 3. The RT-PCR reactions to detect bcl-2 are performed with the primers: 5′-GGTGCCACCTGTGGTCCACCTG-3′ (SEQ ID NO: 21) and 5′-CTTCACTTGTGGCCCAGATAGG-3′ (SEQ ID NO: 22) and 1 μg of input total RNA. Control RT-PCR reactions against β-actin are also performed using the primers 5′-GAGCTGCGTGTGGCTCCCGAGG-3′ (SEQ ID NO: 23) and 5′-CGCAGGATGGCATGGGGGGCATACCCC-3′ (SEQ ID NO: 24) and 0.1 μg of input total RNA.

All bcl-2 and β-actin RT-PCR reactions are performed according to the following program on a PTC-100 thermocycler (MJ Research): Step 1, 50° C. for 35 minutes; Step 2, 94° C for 2 minutes; Step 3, 60° C. for 30 seconds; Step 4, 72° C. for 1 minute; Step 5, 94° C. for 30 seconds; Step 6, go to step 3, 35 more times; Step 7, 72° C. for 10 minutes; Step 8, end.

All RT-PCR products are separated on a 4% Super Resolution Agarose TBE gel and stained with CyberGold™ according to the manufacturer's instructions. Gels are photographed on Polaroid Type 667 film.

### Example 5

### Antisense Targeting of bcl-2A and bcl-xL

Many tumors overexpress multiple chemoresistance genes simultaneously, and are thus unlikely to respond to antisense-based therapies against only one specific chemoresistance gene at a time. Knockout of multiple resistance genes with a single antisense oligonucleotide can enhance chemosensitization in resistant tumors. A known example of such simultaneous expression of chemoresistance genes is bcl-2A and bcl-xL which are distinct, but related, transforming oncogenes are are overexpressed in many human cancers. Most importantly, the overexpression of both bcl-2 family members has been shown to confer chemoresistance to cells.

Previously reported attempts to knock out both genes simultaneously were based on conventional oligonucleotides that are perfectly complementary to one gene or the other, but not both, and thus have several mismatches and low activity against one of the target genes. Thus, these attempts have relied on non-specific RNase H-dependent activity of long oligonucleotides. In contrast, the use of two or more oligonucleotides, one targeted against each gene, is far more likely to result in toxic effects and to produce non-specific antisense activity.

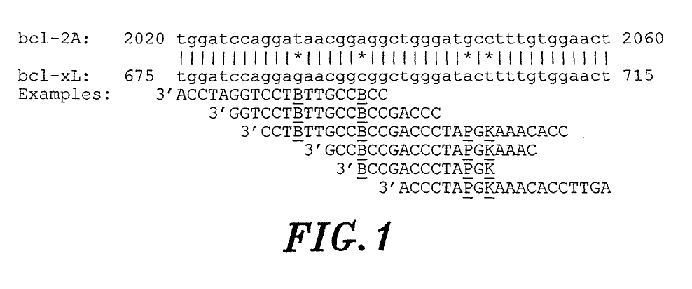

The present invention provides a single antisense oligonucleotide for simultaneous knockout of two or more genes. For example, bcl-2 and bcl-xL are simultaneously targeted with a single oligonucleotide containing one or more universal and/or degenerate bases targeted to the small region of high nucleotide homology shown in FIG. 1. Six representative antisense oligonucleotides containing one or more universal and/or degenerate bases, and the regions to which they hybridize, are shown in FIG. 1. (Human bcl-2 mRNA (HUMBCL2A) - GenBank #M13994; bcl-xL mRNA (HSBCLXL)—GenBank #Z23115) Asterisks indicate mismatches in the region of nucleotide similarity. Base numbers are as defined in GenBank.

### Example 6

### Targeting of Two or More Related Genes

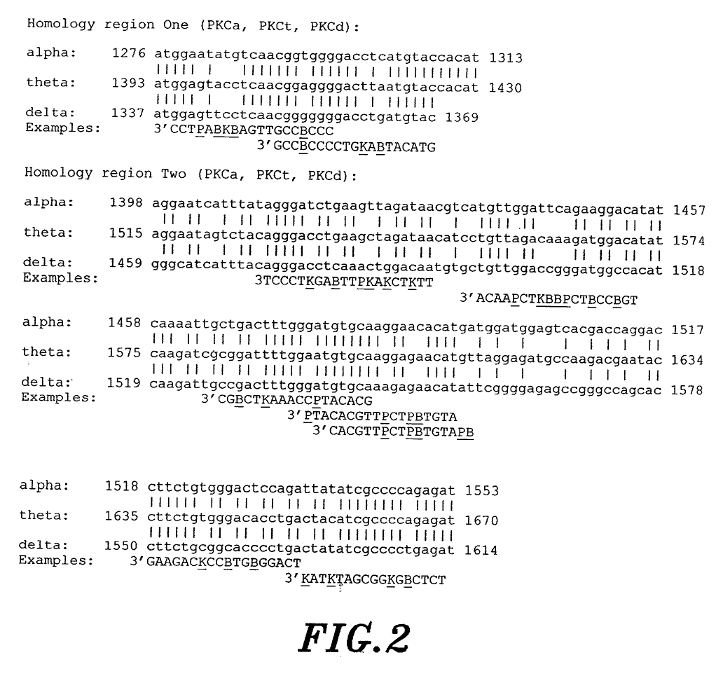

The protein kinase C (PKC) gene family comprises gene products which regulate cell growth by phosphorylating other proteins in response to extracellular signals. Overexpression of PKC genes has been detected in several human tumor types and PKC genes are believed to be potential cancer therapy targets. Despite the similarity of PKC family members at the protein level, the nucleotide sequences can be significantly different. Antisense oligonucleotides including one or more universal or ambiguous bases allows two or more PKC family members to be targeted at the nucleotide level. FIG. 2 shows a sequence alignment of homology regions one and two of human PKCα mRNA (HSPKCA1; GenBank #X52479), human PKCθ mRNA (HUMPKCTH; GenBank #L07860) and human PKCδ mRNA (HUMPKCD13×; GenBank #L07860). Representative oligonucleotides for targeting two or three of these PKC family members are shown in FIG. 2.

### Example 7

### Targeting Two Alleles of the Same Gene

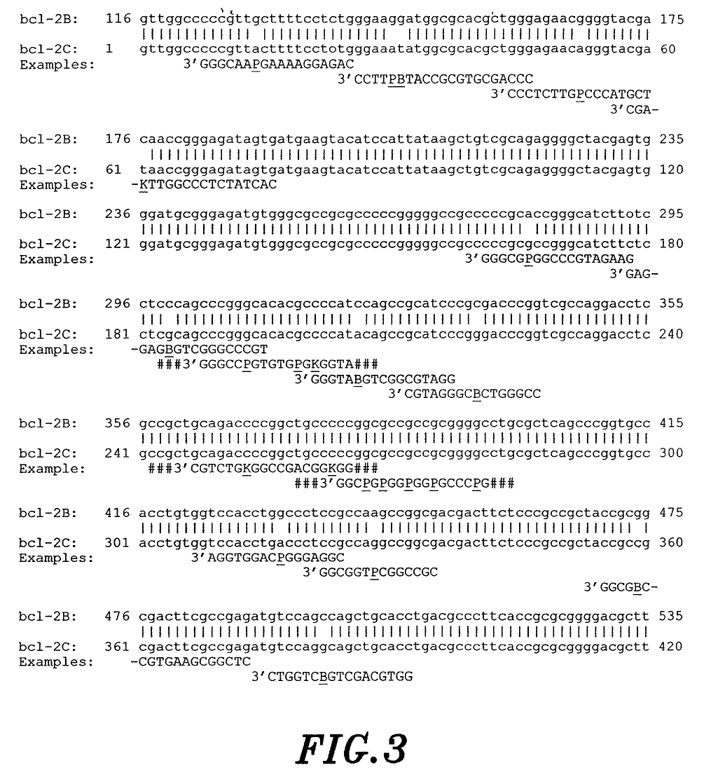

Comparison of allelic variations is an important human oncogene, bcl-2, reveals several single nucleotide polymorphisms (SNPS) within the general human population. Overexpression of any known allele of bcl-2 has been shown to confer chemoresistance in human tumors and is regarded as a poor prognostic indicator. Two or more alleles of the bcl-2 gene can be targeted with single oligonucleotides including one or more universal or degenerated bases without restriction by the occurrence of SNPs. The two regions of human bcl-2B (HUMBCL2B; GenBank #M13995) and human bcl-2C (HUMBCL2C; GenBank #M14745) are shown in FIG. 3, as are representative oligonucleotides which target regions of both alleles.

This allows an antisense oligonucleotide gene walk, the evaluation of a series of antisense oligonucleotides distributed throughout the entire length of overlap between the genetic alleles, to be performed without limitation by the occurrence of SNPs. If SNPs could not be included in the regions targeted by antisense oligonucleotides, the gene walk would be far less effective at identifying effective antisense target sites that yield efficient inhibition of gene expression.

### Example 8

### Elimination of Problematic Antisense Base Sequence Motifs

The oligonucleotides flanked by “###” in FIG. 3 illustrate another advantage of incorporation of universal and/or degenerate bases into antisense oligonucleoitdes, namely the elimination of “CG” dinucleotides and tetra-G sequences which can have deleterious effects as previously discussed. Thus, the use of universal and/or degenerate bases eliminates sequence-dependent, non-antisense effects by substituting universal and/or ambiguous bases into problematic sequence motifs. This is also illustrated below:

| | | | |
|---|---|---|---|
| Anti-bcl-2: | 3′GGGCCCGTGTGCGGGGTA | (SEQ ID NO:25) | (tetra-G) |
| becomes: | 3′-GGGCCPGTGTGPGKGGTA | (SEQ ID NO:26) | |
| Anti-bcl-2: | 3′-CGTCTGGGGCCGACGGGGG | (SEQ ID NO:27) | (double tetra-G) |
| becomes: | 3′-CGTCTGKGGCCGACGGKGG | (SEQ ID NO:28) | |
| Anti-bcl-2: | 3′-GGCCGCGGCGGCGCCCCG | (SEQ ID NO:29) | (highly CG) |
| becomes: | 3′-GGCPGPGGPGGPGCCCPG | (SEQ ID NO:30) | |

While particular embodiments of the invention have been described in detail, it will be apparent to those skilled in the art that these embodiments are exemplary rather than limiting, and the true scope of the invention is that defined in the following claims.

What is claimed is:

1. An antisense oligonucleotide comprising at least one non-naturally occurring backbone linkage and between 6 and about 50 bases, wherein at least one of said bases are universal and/or degenerate bases and, wherein said antisense oligonucleotide complements at least two RNA molecules of a different sequence.

2. The antisense oligonucleotide of claim 1, wherein no more than about 50% of said bases are universal and/or degenerate bases.

3. An antisense oligonucleotide comprising a first non-RNase H recruiting region of between 3 and about 15 bases, an RNase H recruiting region of between 3 and about 15 bases, and a second non-RNase H recruiting region, wherein at least one of said bases are universal and/or degenerate bases and, wherein said antisense oligonucleotide complements at least two RNA molecules of a different sequence.

4. The antisense oligonucleotide of claim 3, wherein no more than about 50% of said bases are universal and/or degenerate bases.

5. An antisense oligonucleotide comprising a non-RNase H recruiting section and an RNase H recruiting section, wherein at least one of said bases are universal and/or degenerate bases and, wherein said antisense oligonucleotide complements at least two RNA molecules of a different sequence.

6. The antisense oligonucleotide of claim 5, wherein no more than about 50% of said bases are universal and/or degenerate bases.

7. An antisense oligonucleotide comprising an RNase L-recruiting region comprising a 2′-5′ adenosine oligomer, wherein an RNA targeting region of said antisense oligonucleotide comprises at least one universal and/or degenerate bases and, wherein said antisense oligonucleotide complements at least two RNA molecules of a different sequence.

8. The antisense oligonucleotide of claim 7, wherein said RNA targeting region comprises no more than about 50% universal and/or degenerate bases.

9. An antisense oligonucleotide comprising an RNase P-recruiting region, wherein an RNA targeting region of said antisense oligonucleotide comprises at least one universal and/or degenerate bases and, wherein said antisense oligonucleotide complements at least two RNA molecules of a different sequence.

10. The antisense oligonucleotide of claim 9, wherein said RNA targeting region comprises no more than about 50% universal and/or degenerate bases.

11. A ribozyme comprising an RNA targeting region, which comprises at least one universal and/or degenerate bases, wherein said antisense oligonucleotide complements at least two RNA molecules of a different sequence.

12. The ribozyme of claim 11, wherein said RNA targeting region comprises no more than about 50% universal and/or degenerate bases.

13. A method of cleaving a plurality of target RNA molecules of different sequence, comprising contacting said target RNA molecules with an antisense oligonucleotide according to any one of claims 1-10 in the presence of an RNAse capable of cleaving said target RNA molecules.

14. The method of claim 13, wherein said RNase is selected from the group consisting of RNAse H, RNAse L, and RNAse P.

15. A method of cleaving a plurality of target RNA molecules of different sequence, comprising contacting said target RNA molecules with a ribozyme according to claims 11 or 12.

16. A method of cleaving a plurality of target RNA molecules of different sequence, comprising contacting said target RNA molecules with an antisense oligonucleotide comprising between 6 and -about 50 bases, wherein said antisense oligonucleotide comprises at least one universal and/or degenerate base and, wherein said antisense oligonucleotide complements at least two RNA molecules of a different sequence.

17. A method for reducing the deleterious effects of an antisense oligonucleotide comprising one or more sequence motifs, comprising replacing one or more bases within said one or more sequence motifs with one or more universal and/or degenerate bases.

18. The method of claim 17, wherein said sequence motif is a CG dinucleotide.

19. The method of claim 17, wherein said sequence motif is a poly-G sequence.


SEQUENCE LISTING (30 sequences)

SEQ ID NO: 1 - DNA, 18 bp, Artificial Sequence
  Synthetic oligonucleotide primers
nnnnnnnnnnnnnnnnnn

SEQ ID NO: 2 - DNA, 18 bp, Artificial Sequence
  Synthetic oligonucleotide primers
nnnnnnnnnnnnnnnnnn

SEQ ID NO: 3 - DNA, 18 bp, Artificial Sequence
  Synthetic oligonucleotide primers
nnnnnnnnnnnnnnnnnn

SEQ ID NO: 4 - DNA, 18 bp, Artificial Sequence
  Synthetic oligonucleotide primers
nnnnnnnnnnnnnnnnnn

SEQ ID NO: 5 - DNA, 18 bp, Artificial Sequence
  Synthetic oligonucleotide primers
nnnnnnnnnnnnnnnnnn

SEQ ID NO: 6 - DNA, 18 bp, Artificial Sequence
  Synthetic oligonucleotide primers
nnnnnnnnnnnnnnnnnn

SEQ ID NO: 7 - DNA, 18 bp, Artificial Sequence
  Synthetic oligonucleotide primers
nnnnnnnnnnnnnnnnnn

SEQ ID NO: 8 - DNA, 18 bp, Artificial Sequence
  Synthetic oligonucleotide primers
nnnnnnnnnnnnnnnnnn

SEQ ID NO: 9 - DNA, 18 bp, Artificial Sequence
  Synthetic oligonucleotide primers
nnnnnnnnnnnnnnnnnn

SEQ ID NO: 10 - DNA, 18 bp, Artificial Sequence
  Synthetic oligonucleotide primers
nnnnnnnnnnnnnnnnnn

SEQ ID NO: 11 - DNA, 18 bp, Artificial Sequence
  Synthetic oligonucleotide primers
nnnnnnnnnnnnnnnnnn

SEQ ID NO: 12 - DNA, 22 bp, Artificial Sequence
  Synthetic oligonucleotide primers
cgaaattaaatcgactcactat

SEQ ID NO: 13 - DNA, 80 bp, Artificial Sequence
  Synthetic oligonucleotide primers
caggcccagcagcagcagcagcagcagcatggtgggcgaattcgcgattcgaagccctatagtgagtcgtattaatttcg

SEQ ID NO: 14 - DNA, 22 bp, Artificial Sequence
  Synthetic oligonucleotide primers
ggttctcctcctcaactgggat

SEQ ID NO: 15 - DNA, 76 bp, Artificial Sequence
  Synthetic oligonucleotide primers
ggttctcctcctcaactgggatgatgcccagggagagctgtagcctcaggcccagcagcagcagcagcagcagcat

SEQ ID NO: 16 - DNA, 100 bp, Artificial Sequence
  Synthetic oligonucleotide primers
gggcttcgaatcgcgaattcgcccaccatgctgctgctgctgctgctgggcctgaggctacagctctccctgggcatcatcccagttgaggaggagaacc

SEQ ID NO: 17 - DNA, 18 bp, Artificial Sequence
  Synthetic oligonucleotide primers
gttctcbbbbbbgagttt

SEQ ID NO: 18 - DNA, 20 bp, Artificial Sequence
  Synthetic oligonucleotide primers
gttctcgctggtgagtttca

SEQ ID NO: 19 - DNA, 18 bp, Artificial Sequence
  Synthetic oligonucleotide primers
tctbccbbcbtbcbccbt

SEQ ID NO: 20 - DNA, 18 bp, Artificial Sequence
  Synthetic oligonucleotide primers
tctcccagcgtgcgccat

SEQ ID NO: 21 - DNA, 22 bp, Artificial Sequence
  Synthetic oligonucleotide primers
ggtgccacctgtggtccacctg

SEQ ID NO: 22 - DNA, 22 bp, Artificial Sequence
  Synthetic oligonucleotide primers
cttcacttgtggcccagatagg

SEQ ID NO: 23 - DNA, 22 bp, Artificial Sequence
  Synthetic oligonucleotide primers
gagctgcgtgtggctcccgagg

SEQ ID NO: 24 - DNA, 26 bp, Artificial Sequence
  Synthetic oligonucleotide primers
cgcaggatggcatggggggcataccc

SEQ ID NO: 25 - DNA, 18 bp, Artificial Sequence
  Synthetic oligonucleotide primers
gggcccgtgtgcggggta

SEQ ID NO: 26 - DNA, 18 bp, Artificial Sequence
  Synthetic oligonucleotide primers
gggccngtgtgngnggta

SEQ ID NO: 27 - DNA, 19 bp, Artificial Sequence
  Synthetic oligonucleotide primers
cgtctggggccgacggggg

SEQ ID NO: 28 - DNA, 19 bp, Artificial Sequence
  Synthetic oligonucleotide primers
cgtctgkggccgacggkgg

SEQ ID NO: 29 - DNA, 18 bp, Artificial Sequence
  Synthetic oligonucleotide primers
ggccgcggcggcgccccg

SEQ ID NO: 30 - DNA, 18 bp, Artificial Sequence
  Synthetic oligonucleotide primers
ggcngnggnggngcccng